(19) United States
(12) Patent Application Publication
(10) Pub. No.: US 2008/0058305 A1
(43) Pub. Date: Mar. 6, 2008

(54) Processes for the purification of (3R,4S)-4-(4-hydroxy-protected-phenyl)-1-(4-fluorophenyl)-3-[3-(4-fluorophenyl)-3-oxopropyl]azetidin-2-one

(76) Inventors: Vinod Kumar Kansal, Haryana (IN); Suhail Ahmad, New Delhi (IN); Sankaran Sethumadhavan, Gurgaon (IN); Shanmugavel Mariappan, Tamilnadu (IN)

Correspondence Address: KENYON & KENYON LLP, ONE BROADWAY, NEW YORK, NY, 10004, US

(21) Appl. No.: 11/786,448

(22) Filed: Apr. 10, 2007

Related U.S. Application Data
(60) Provisional application No. 60/841,160, filed on Aug. 29, 2006.
(60) Provisional application No. 60/897,360, filed on Jan. 24, 2007.

(51) Int. Cl. A61K 31/397; A61P 3/00; C07D 205/04

(52) U.S. Cl. 514/210.02; 540/200

(57) ABSTRACT

Provided are processes for purifying (3R,4S)-4-(4-hydroxyprotected-phenyl)-1-(4-fluorophenyl)-3-[3-(4-fluorophenyl)-3-oxopropyl]azetidin-2-one having the following formula II

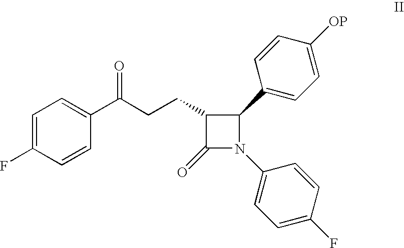

wherein X and Y are hydrogen or a substituted or unsubstituted C₁₋₈ alkyl; n is an integer between 0 and 3; and P is a hydroxyl protecting group. The Compound of formula II may be converted to an azetidinone compound, which is useful, for example, in reducing cholesterol in mammals.

### CROSS REFERENCE TO RELATED APPLICATIONS

This application claims the benefit of U.S. Ser. No. 60/841,160, filed Aug. 29, 2006, and Ser. No. 60/897,360, filed Jan. 24, 2007, the contents of both of which are incorporated herein by reference in their entirety.

### FIELD OF THE INVENTION

The present invention relates to processes for the purification of (3R,4S)-4-(4-hydroxyprotected-phenyl)-1-(4-fluorophenyl)-3-[3-(4-fluorophenyl)-3-oxopropyl]azetidin-2-one, an intermediate for the preparation of ezetimibe.

### BACKGROUND OF THE INVENTION

Hydroxyl-alkyl substituted azetidinones useful as hypocholesterolemic agents in the treatment and prevention of atherosclerosis include 1-(4-fluorophenyl)-3(R)-[3-(4-fluorophenyl)-3(S)-hydroxypropyl]-4(S)-(4-hydroxyphenyl)-2-azetidinone, i.e. ezetimibe. Ezetimibe is a selective inhibitor of intestinal cholesterol and related phytosterol absorption. The empirical formula for ezetimibe is C₂₄H₂₁F₂NO₃, and its molecular weight is 409.4 g/mol. Ezetimibe is a white, crystalline powder that is freely to very soluble in ethanol, methanol and acetone and practically insoluble in water. Ezetimibe has the following chemical structure:

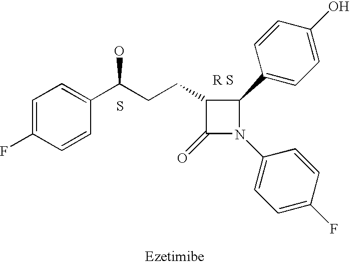

Ezetimibe is sold under the name ZETIA® by Merck/Schering-Plough Pharmaceuticals, and is approved by the United States Food and Drug Administration for use in patients with high cholesterol to reduce LDL cholesterol and total cholesterol.

Processes for preparing 1-(4-fluorophenyl)-3(R)-[3-(4-fluorophenyl)-3(S)-hydroxypropyl]-4(S)-(4-hydroxyphenyl)-2-azetidinone (ezetimibe) using an intermediate Compound II, below, are disclosed in U.S. Pat. Nos. 5,631,365; 5,739,321 and 5,886,171.

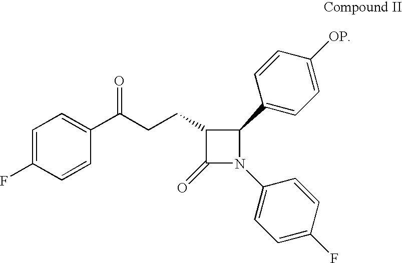

The process disclosed in these references involve a multi step procedure starting either from methyl-4-(chloroformyl)butyrate in U.S. Pat. No. 5,631,365 or 3(S)-Hydroxy-γ-butyrolactone in U.S. Pat. Nos. 5,739,321 and 5,886,171. The product from the steps described are typically in liquid form. Because the conversion of these starting materials to Compound of formula II involve the use of many reagents and/or catalysts e.g., viz. lithium N,N-diisopropylamide (LDA), enolether, (Ph₃)₃RhCl, tetrakis triphenyl Pd(0), Grignard/Zincate etc., the Compound of formula II produced therefrom is generally of lower purity.

There is a need in the art for commercially viable and plant-friendly purification and crystallization processes for Compound II.

### SUMMARY OF THE INVENTION

In one embodiment, the invention encompasses a process for purifying (3R,4S)-4-(4-hydroxyprotected-phenyl)-1-(4-fluorophenyl)-3-[3-(4-fluorophenyl)-3-oxopropyl]azetidin-2-one (“the Compound of formula II”) having the following formula II

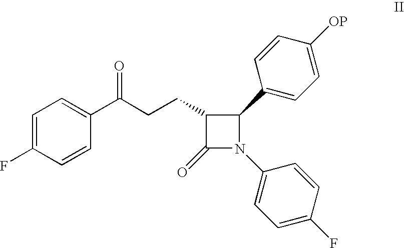

comprising the steps of:

(a) reacting the Compound of formula II with a diol derivative of formula III:

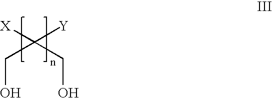

in the presence of at least one acid catalyst selected from the group consisting of: sulfonic acid, para-toluene sulfonic acid, methane sulfonic acid, benzene sulfonic acid, a C₁₋₄ tri-alkylsilyl chloride, titanium tetrachloride, titanium tetra isopropoxide, aluminum chloride, zinc chloride, and BF₃ etherate, or at least one salt of an organic base selected from the group consisting of: pyridinum hydrobromide, pyridinum hydrochloride, pyridinum hydroiodide, pyridinum paratoluenesulfonate, pyridinum methane sulfonate, pyridinum benzene sulfonate, C₁₋₄ tri-alkyl amine hydrochloride, C₁₋₄ tri-alkyl amine hydrobromide, and C₁₋₄ tri-alkyl amine hydroiodide, to obtain a Compound of formula IV:

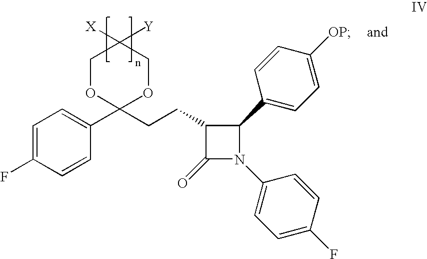

(b) adding an acid to obtain the Compound of formula II, wherein X and Y are hydrogen or a substituted or unsubstituted C₁₋₈ alkyl; n is an integer between 0 and 3; and P is a hydroxyl protecting group.

In further embodiment, the invention encompasses the Compound of formula II prepared according to a process of the present invention. Preferably, the purity of the Compound of formula II is at least about 95%, preferably at least about 99%, more preferably at least about 99.8%, and more preferably at least about 99.9% by percent weight HPLC.

The invention also encompasses a Compound of formula II having a purity of at least about 95%, preferably at least about 99%, more preferably at least about 99.8%, and more preferably at least about 99.9% by percent weight HPLC. The invention further encompasses a Compound of formula II having an assay of at least about 98%, preferably at least about 99%, more preferably at least about 99.8%, and more preferably at least about 99.9% by HPLC.

The invention further encompasses a process for preparing a Compound of formula IV:

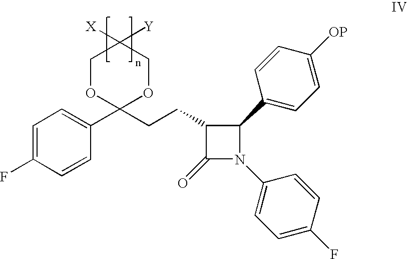

wherein X and Y are hydrogen or a substituted or unsubstituted C₁₋₈ alkyl; n is an integer between 0 and 3; and P is a hydroxyl protecting: group. The process comprises reacting the Compound of formula II with a diol derivative of formula III as described above in the presence of at least one acid catalyst selected from the group consisting of: sulfonic acid, para-toluene sulfonic acid, methane sulfonic acid, benzene sulfonic acid, a C₁₋₄ tri-alkylsilyl chloride, titanium tetrachloride, titanium tetra isopropoxide, aluminum chloride, zinc chloride, and BF₃ etherate, or at least one salt of an organic base selected from the group consisting of: pyridinum hydrobromide, pyridinum hydrochloride, pyridinum hydroiodide, pyridinum paratoluenesulfonate, pyridinum methane sulfonate, pyridinum benzene sulfonate, triethyl amine hydrochloride, pyridinum C₁₋₄ tri-alkyl amine hydrochloride, pyridinum C₁₋₄ tri-alkyl amine hydrobromide, and pyridinum C₁₋₄ tri-alkyl amine hydroiodide to obtain the compound formula IV.

The invention also encompasses a process for preparing a Compound of formula II comprising adding an acid to the Compound of formula IV to form the Compound of formula II.

In another embodiment, the invention encompasses a process for preparing an azetidinone compound comprising preparing the Compound of formula IV according to a process of the present invention, and converting the Compound of formula IV to an azetidinone compound. The invention also encompasses a process for preparing an azetidinone compound comprising preparing the Compound of formula II according to a process of the present invention, and converting the Compound of formula II to an azetidinone compound. Preferably, the azetidinone is ezetimibe.

The invention also encompasses an azetidinone, preferably ezetimibe, prepared according to the processes of the invention, pharmaceutical compositions comprising such azetidinone, e.g., ezetimibe, and methods for reducing cholesterol in a mammal comprising administering a therapeutically effective amount of the azetidinone, e.g., ezetimibe, or a pharmaceutical composition of the invention.

### DETAILED DESCRIPTION OF THE INVENTION

As used herein, the term “alkyl” refers to a straight or branched hydrocarbon chain radical consisting of carbon and hydrogen atoms, containing no unsaturation, having from one to eight, preferably one to six, and more preferably one to four carbon atoms, and which is attached to the rest of the molecule by a single bond, e.g., methyl, ethyl, n-propyl, 1-methylethyl (isopropyl), n-butyl, n-pentyl, 1,1-dimethylethyl (t-butyl), or the like.

As used herein, the term “halogenated hydrocarbons” refers to cyclic or acyclic, saturated or unsaturated aliphatic (e.g., C₁₋₈, preferably C₁₋₆, more preferably C₁₋₄) or aromatic (e.g., C₆₋₁₂, preferably C₆₋₁₀, more preferably C₆₋₈) hydrocarbons. Examples of halogenated hydrocarbons include, but are not limited to, halogenated alkanes such as chloromethane, dichloromethane, trichloromethane, chloroethane, 1,1- or 1,2-dichloroethane, trichloroethane, dichlorotrifluoroethane, difluoroethane, hexachloroethane, pentafluoroethane, halogenated alkenes such as such as tetrachloroethene, dichloroethene, trichloroethene, vinyl chloride, chloro-1,3-butadiene, chlorotrifluoroethylene, or halogenated benzenes such as benzotrichloride, benzyl chloride, bromobenzene, chlorobenzene, chlorotoluene, dichlorobenzene, fluorobenzene, or trichlorobenzene. A preferred halogen is chlorine. Preferred halogenated hydrocarbons include halogenated aromatic hydrocarbons or halogenated C₁-C₄ alkanes, and more preferably chlorinated aromatic hydrocarbons or chlorinated C₁-C₄ alkanes. The more preferred halogenated hydrocarbons include chlorobenzene, o- or p-dichlorobenzene, dichloromethane, or o-chlorotoluene.

The term “substituted” (e.g., as used in “substituted alkyl”) refers to the replacement of one or more, preferably from one to three, hydrogen atoms with one or more substituents, examples of which include hydroxy, carboxyl, alkyl (e.g., C₁ to C₆), alkoxy (e.g., C₁ to C₆), aryl (e.g., C₆ to C₁₄), arylalkyl (C₇₋₁₅), cycloalkyl (C₃ to C₁₂), amino, or the like. Further, such substituents may include such groups as alkylthio (e.g., C₁ to C₆), nitro, halo, cyano, haloalkyl (e.g., C₁ to C₆), haloalkoxy (e.g., C₁ to C₆), carboxamido, mono(C₁ to C₆ alkyl)amino, di(C₁ to C₆ alkyl)amino, C₁ to C₆ alkylsulfonylamino, or the like. The substituents may be the same or different.

In U.S. Pat. Nos. 5,631,365; 5,739,321 and 5,886,171, the Compound of formula II is prepared by methods known in the art generally resulting in lower purity and/or lower yield compared to those made according to the invention.

The invention encompasses a process for purifying (3R,4S)-4-(4-hydroxyprotected-phenyl)-1-(4-fluorophenyl)-3-[3-(4-fluorophenyl)-3-oxopropyl]azetidin-2-one (“the Compound of formula II”). In one embodiment, the Compound of formula II is purified by:
(a) reacting the Compound of formula II with a diol derivative of formula III having the following structure:

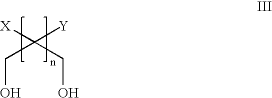

in the presence of at least one acid catalyst selected from the group consisting of sulfonic acid, para-toluene sulfonic acid, methane sulfonic acid, benzene sulfonic acid, a C₁₋₄ tri-alkylsilyl chloride, e.g., trimethylsilyl chloride (“TMSCl”), titanium tetrachloride, titanium tetra isopropoxide, aluminum chloride, zinc chloride, and BF₃ etherate; or at least one salt of an organic base selected from the group consisting of: pyridinum hydrobromide (“PY:HBr”), pyridinum hydrochloride (“PY:HCl”), pyridinum hydroiodide (“PY:HI”), pyridinum paratoluenesulfonate (“PPTS”), pyridinum methane sulfonate, pyridinum benzene sulfonate, C₁₋₄ tri-alkyl amine hydrochloride, e.g., triethyl amine hydrochloride, C₁₋₄ tri-alkyl amine hydrobromide, e.g., triethyl amine hydrobromide, and C₁₋₄ tri-alkyl amine hydroiodide, e.g., triethyl amine hydroiodide, to obtain a Compound of formula IV, as illustrated in the following scheme:

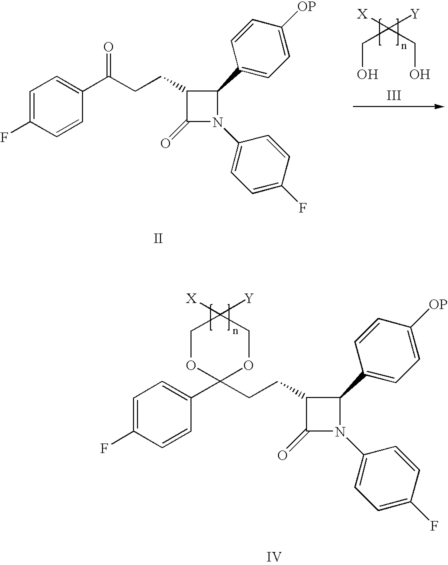

; and
(b) adding at least one acid to obtain the Compound of formula II, as illustrated in the following scheme:

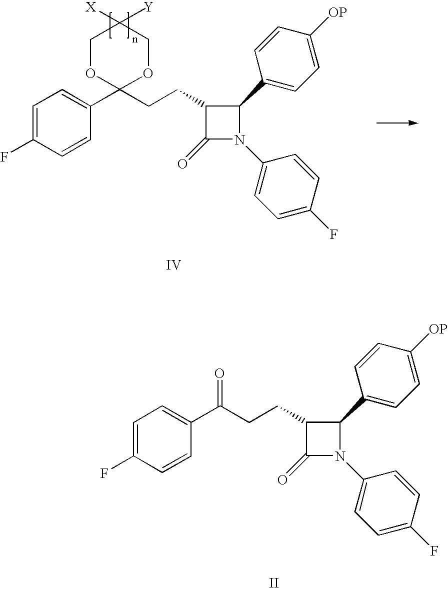

wherein X and Y are hydrogen or a substituted or unsubstituted C₁₋₈ alkyl, preferably C₁₋₆ alkyl, more preferably C₁₋₄ alkyl, and may be the same or different; n is an integer between 0 and 3; and P is a hydroxyl protecting group.

A suitable protecting group, or “P”, for hydroxy functionalities, includes acyl, tetrahydropyranyl, tetrahydrothiopyranyl, tetrahydrofuranyl, tetrahydrothiofuranyl, 2-(phenylselenyl)ethyl, o-nitrobenzyl, benzyl, p-methoxy benzyl, tri(C₁₋₆ alkyl)silyl (where the (C₁₋₆ alkyl) groups may be the same or different), e.g., trimethylsilyl, triisopropylsilyl, isopropyldimethylsilyl, and t-butyldimethylsilyl, and tri(C₆₋₁₀ aryl)silyl (where the (C₆₋₁₀ aryl) groups may be the same or different), e.g., t-butyldiphenylsilyl, tribenzylsilyl, acetyl, isobutyl, pivaloyl, adamantoyl, benzoyl, 2,4,6-trimethylbenzoly (mesitoyl), methyl carbonyl, p-nitrophenyl carbonyl, p-nitrobenzyl carbonyl, S-benzyl thiocarbonyl, and N-phenylcarboyl. A preferred protecting group is where P is selected from the group consisting of: benzyl, trimethylsilyl, para-methoxy benzyl, acetyl, and methyl. A more preferred protecting group is benzyl.

Preferably, X and Y are both hydrogen or C₁₋₈ alkyl, and more preferably C₁₋₄ alkyl, most preferably methyl. Preferably, n is one. Preferably, the diol derivative of formula III is neopentyl glycol, propane-1,3-diol, or ethylene glycol, and more preferably neopentyl glycol. Preferably, the Compound of formula IV is 4(S)-(4-Benzyloxyphenyl)-1-(4-fluorophenyl)-3(R)-{2-[2-(4-fluorophenyl)-5,5-dimethyl-[1,3]-dioxan-2-yl]-ethyl}azetidine-2-one.

The acid catalyst may be selected from the group consisting of: sulfonic acid, para-toluene sulfonic acid, methane sulfonic acid, benzene sulfonic acid, trimethyl silyl chloride (TMSCl), titanium tetrachloride, titanium tetra isopropoxide, aluminum chloride, zinc chloride, BF₃-etherate, and mixtures thereof. Preferably, the acid catalyst is selected from the group consisting of para-toluene sulfonic acid, trimethyl silyl chloride, and BF₃ etherate.

The salt of the organic base may be selected from the group consisting of: pyridinum hydrobromide (“PY:HBr”), pyridinum hydrochloride (“PY:HCl”), pyridinum hydroiodide (“PY:HI”), pyridinum paratoluenesulfonate (“PPTS”), pyridinum methane sulfonate, pyridinum benzene sulfonate, C₁₋₄ tri-alkyl amine hydrochloride, e.g., triethyl amine hydrochloride, C₁₋₄ tri-alkyl amine hydrobromide, e.g., triethyl amine hydrobromide, C₁₋₄ tri-alkyl amine hydroiodide, e.g., triethyl amine hydroiodide, and mixtures thereof. Preferably, the salt of an organic base is selected from the group consisting of pyridinum hydrobromide and pyridinum paratoluenesulfonate.

Preferably, the acid is selected from the group consisting of: a mineral acid, e.g., phosphoric acid, hydrobromic acid, hydrochloric acid, or sulfuric acid, a C₂₋₆ carboxylic acid, e.g., acetic acid, formic acid, or propionic acid, camphor sulfonic acid, and mixtures thereof. More preferably, the acid is selected from the group consisting of: formic acid, camphor sulfonic acid, and sulfuric acid. Most preferably, the acid is selected from the group consisting of: formic acid and sulfuric acid.

Optionally, the reaction of step (a) may further comprise adding at least one organic solvent. If an organic solvent is used, the organic solvent is preferably selected from the group consisting of a halogenated hydrocarbon (e.g., C₁ to C₈), aromatic hydrocarbon (e.g., C₆ to C₁₄), aliphatic cyclic hydrocarbons (e.g., C₁ to C₈) and mixtures thereof. Preferably, the halogenated hydrocarbon is selected from the group consisting of dichloromethane and dichloroethane. The preferred halogen is chlorine. The preferred halogenated hydrocarbons are halogenated aromatic hydrocarbons or C₁-C₄ halogenated alkanes, and more preferably, chlorinated aromatic hydrocarbons or C₁-C₄ halogenated alkanes. The more preferred halogenated hydrocarbons are chlorobenzene, o- or p-dichlorobenzene, dichloromethane, or o-chlorotoluene. Preferably, the aromatic hydrocarbon is toluene or xylene. Preferably, the aliphatic cyclic hydrocarbon is cyclohexane or cyclopentane.

Optionally, the reaction of step (a) may further comprise adding at least one organic solvent. If an organic solvent is used, the organic solvent in step (b) is preferably selected from the group consisting of a C₆ to C₁₄ aromatic hydrocarbon, a C₁ to C₅ alcohol, a C₂ to C₇ ester, a C₄ to C₇ ether, halogenated hydrocarbons (e.g., C₁ to C₈, preferably C₁ to C₃), and mixtures thereof. Preferably, the C₆ to C₁₄ aromatic hydrocarbon is toluene or xylene. Preferably, the C₁ to C₅ alcohol is methanol, ethanol, or propanol. Preferably, the C₂ to C₇ ester is propanoate, ethyl acetate, or propyl acetate. Preferably, the C₄ to C₇ ether is tetrahydrofuran (THF) or methyl tert butyl ether (MTBE). Preferably, the C₂ to C₃ halogenated hydrocarbon is dichloromethane. Most preferably, the organic solvent is ethanol, propanol or dichloromethane.

The invention also encompasses the Compound of formula II prepared according to a process of the present invention. Preferably, the process produces the Compound of formula II at a yield of at least about 80% by weight, and more preferably at least about 87% by weight. Preferably, the purity of the Compound of formula II is at least about 95%, preferably at least about 99%, more preferably at least about 99.8%, and more preferably at least about 99.9% by percent weight HPLC.

In one embodiment, the invention encompasses a Compound of formula II having a purity of at least about 95%, preferably at least about 99%, more preferably at least about 99.8%, and more preferably at least about 99.9% by percent weight HPLC. As used herein, “percent by weight HPLC” reflects the peak area divided by the total area of all peaks in a sample.

In one embodiment, the invention encompasses a Compound of formula II having an assay of at least about 98%, preferably at least about 99%, more preferably at least about 99.8%, and more preferably at least about 99.9% by HPLC. As used herein, the term “assay” refers to the amount of the compound compared to the amount of impurities present. Assay can be determined, e.g., by HPLC, such as a method set forth in the Examples.

The invention also encompasses a process for preparing a Compound of formula IV using a Compound of formula II. In one embodiment, the process comprises reacting the Compound of formula II with a diol derivative of formula III as described above in the presence of at least one acid catalyst selected from the group consisting of: sulfonic acid, para-toluene sulfonic acid, methane sulfonic acid, benzene sulfonic acid, a C₁₋₄ tri-alkylsilyl chloride, titanium tetrachloride, titanium tetra isopropoxide, aluminum chloride, zinc chloride, and BF₃ etherate, or at least one salt of an organic base selected from the group consisting of: pyridinum hydrobromide, pyridinum hydrochloride, pyridinum hydroiodide, pyridinum paratoluenesulfonate, pyridinum methane sulfonate, pyridinum benzene sulfonate, pyridinum C₁₋₄ tri-alkyl amine hydrochloride, pyridinum C₁₋₄ tri-alkyl amine hydrobromide, and pyridinum C₁₋₄ tri-alkyl amine hydroiodide to obtain the compound formula IV. The preferred substituents, solvents, reagents, and/or reaction conditions are as set forth above.

The invention also encompasses a process for preparing a Compound of formula II comprising adding an acid, preferably an acid is selected from the group consisting of formic acid, acetic acid, propionic acid, camphor sulfonic acid, hydrochloric acid, sulfuric acid, mineral acid, a C₂₋₆ carboxylic acid, phosphoric acid, hydrobromic acid, and mixtures thereof, to the Compound of formula IV to form the Compound of formula II. The preferred substituents, solvents, reagents, and/or reaction conditions are as set forth above.

The invention encompasses a process for preparing an azetidinone compound comprising preparing Compound of formula II according to a process of the present invention, and converting Compound of formula II to an azetidinone compound. The invention also encompasses a process for preparing an azetidinone compound comprising preparing the Compound of formula IV according to a process of the present invention, and converting the Compound of formula IV to an azetidinone compound. As used herein, an azetidinone compound is a four membered optionally substituted lactam ring compound having the following structure:

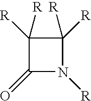

### A preferred azetidinone is ezetimibe.

The Compound of formula IV may be converted to the Compound of formula II using a process described herein, and the Compound of formula II may be converted to an azetidinone according to processes known in the art. For example, the Compound of formula II may be converted to ezetimibe according to the process described in U.S. pending Application Ser. Nos. 60/791,114, and 60/831,908, the contents of each of which are incorporated herein by reference in their entirety.

For example, according to WO 07/030721, ezetimibe can be prepared by reducing (3R,4S)-4-((4-benzyloxy)phenyl)-1-(4-fluorophenyl)-3-(3-(4-fluorophenyl)-3-oxopropyl)-2-azetidinone (Compound II where P=benzyl) with borane dimethyl sulfide complex or borane tetrahydrofuran complex in tetrahydrofuran in the presence of Corey's reagent and subsequently deprotecting the benzyl group, as shown below:

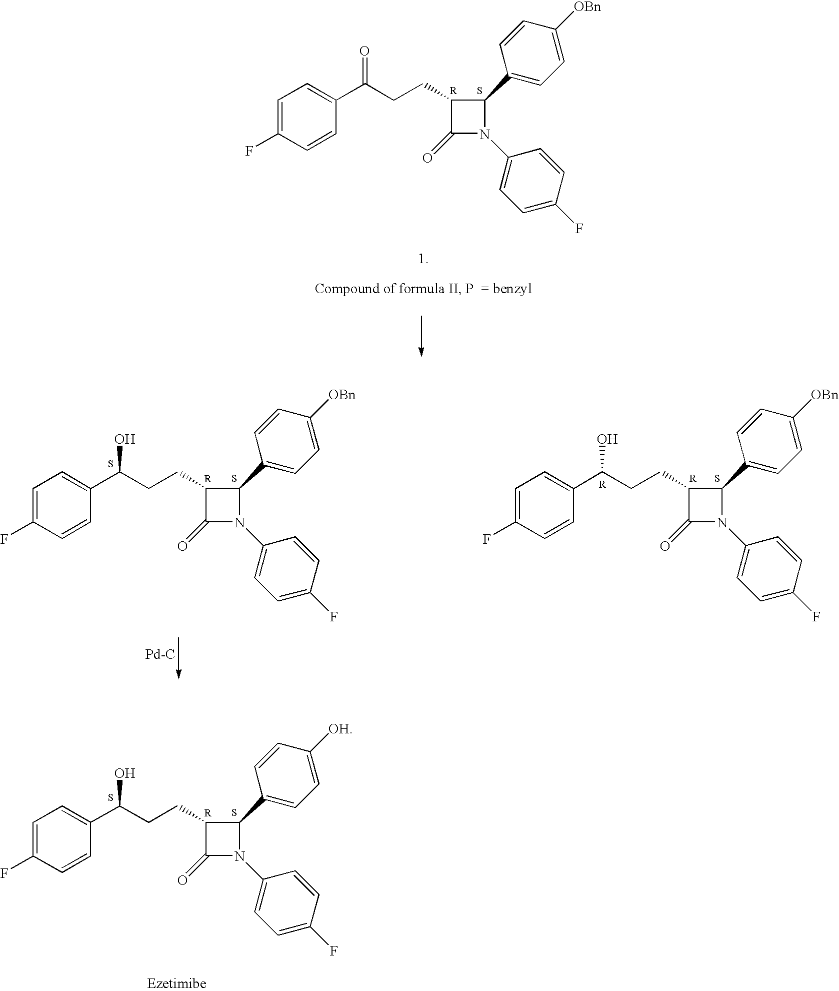

The invention also encompasses an azetidinone, including ezetimibe, prepared according to a process of the invention. The invention further encompasses a pharmaceutical composition comprising such azetidinone (e.g., ezetimibe), and a method for reducing cholesterol in a mammal using such pharmaceutical composition or such azetidinone.

The ezetimibe of the invention herein can be formulated into a variety of compositions for administration to humans and animals for treating diseases through the reduction of cholesterol.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of ezetimibe and/or other active ingredients such as HMG-CoA reductase inhibitors. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol and talc.

Carriers for use in the pharmaceutical compositions may include, but are not limited to, lactose, white sugar, sodium chloride, glucose, urea, starch, calcium carbonate, kaolin, crystalline cellulose, silicic acid, and the like.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like. Absorption accelerators may include, but are not limited to, quaternary ammonium base, sodium laurylsulfate, and the like. Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the ezetimibe forms described herein and any other solid ingredients are dissolved or suspended in a liquid carrier, such as water, vegetable oil, alcohol, polyethylene glycol, propylene glycol or glycerin. Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of ezetimibe or pharmaceutically acceptable salt thereof contained in a pharmaceutical composition for reducing cholesterol according to the present invention is not specifically restricted; however, the dose should be sufficient to treat, ameliorate, or reduce the condition. For example, ezetimibe may be present in an amount of about 1% to about 70%.

The dosage of a pharmaceutical composition for reducing cholesterol according to the present invention will depend on the method of use, the age, sex, weight and condition of the patient. Typically, about 1 mg to 200 mg of ezetimibe may be contained in an administration unit form, preferably a 10 mg tablet.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

HPLC purity data of the Compound of formula II (before and after purification) are illustrated in Tables 1 and 2.

### Preparation of the Compound of Formula IV

(“Compound IV,” or 4(S)-(4-benzyloxyphenyl)-1-(4-fluorophenyl)-3(R)-{2-[2-(4-fluorophenyl)-5,5-dimethyl-[1,3]-dioxan-2-yl]-ethyl}azetidine-2-one)

TABLE 1

| | Compound II | | | Compound IV | | |
|---|---|---|---|---|---|---|
| | | HPLC | | | | HPLC |
| | Input | Purity | Assay | Output | Yield | Purity |
| Example No. | (g) | (%) | (%) | (g) | (%) | (%) |
| Example I(a) | 100 | 68.51 | 47.62 | 46 | 77.6 | 98.22 |
| Example I(b) | 50 | 92.22 | 80.20 | 38 | 77.1 | 97.89 |
| Example I(c) | 50 | 92.22 | 80.20 | 37 | 75.2 | 97.98 |
| Example I(d) | 100 | 68.51 | 47.62 | 42.5 | 72.0 | 98.70 |
| Example I(e) | 100 | 92.22 | 80.20 | 70.2 | 71.1 | 97.72 |
| Example I(f) | 50 | 68.51 | 47.62 | 20.5 | 70.4 | 98.20 |
| Example I(g) | 50 | 92.22 | 80.20 | 34.0 | 68.7 | 97.50 |
| Example I(h) | 50 | 92.22 | 80.20 | 33.6 | 67.6 | 97.02 |

### Preparation of the Compound of Formula II

(“Compound II,” P =benzyl. 4(S)-(4-Benzyloxyphenyl)-1-(4-fluorophenyl)-3(R)-[3-(4-fluorophenyl)-3-oxopropyl]-azetidine-2-one)

TABLE 2

| | Compound IV | | Compound II | | | |
|---|---|---|---|---|---|---|
| | | HPLC | | | HPLC | |
| | Input | Purity | Output | Yield | Purity | Assay |
| Batch No. | (g) | (%) | (g) | (%) | (%) | (%) |
| Example II(a) | 45 | 98.22 | 33 | 87.6 | 99.58 | 99.98 |
| Example II(b) | 37 | 97.89 | 26.5 | 85.8 | 99.20 | 99.85 |
| Example II(c) | 36 | 97.98 | 26.0 | 86.5 | 99.35 | 99.95 |
| Example II(d) | 40 | 98.70 | 29.1 | 86.5 | 99.36 | 99.90 |
| Example II(e) | 70 | 97.72 | 51.0 | 87.5 | 99.45 | 99.88 |
| Example II(f) | 20 | 98.20 | 14.2 | 84.8 | 99.26 | 99.95 |
| Example II(g) | 33 | 97.50 | 23.4 | 85.3 | 99.56 | 99.92 |
| Example II(h) | 33 | 97.02 | 24.0 | 87.9 | 99.42 | 99.97 |

### HPLC Methodology

### Assay of Compound II

Liquid chromatography was carried using the following parameters, and the percentage content of compound of formula II (“Assay”) was calculated from the areas of the peaks and the declared content of compound of formula II.

### Liquid Chromatography:

Test solution (a). Dissolve 25.0 mg of substance to be examined in the mobile phase and dilute to 50.0 ml with the mobile phase.

Standard solution (b) Dissolve 25.0 mg of standard of compound of formula II in the mobile phase and dilute to 50.0 ml with the mobile phase.

| | |
|---|---|
| Column | LUNA C-8(2), (250 × 4.6 mm), 5 μm, |
| | P/N-00G-4249-EO |
| Stationary phase: | Octylsilyl silicagel for chromatography R (5 μm) |
| Mobile phase: | mix 55 volumes of acetonitrile R, 45 volumes |
| | of buffer solution |
| Buffer solution: | dissolve 1.0 mL of phosphoric acid R in 1 liter |
| | of water R and adjust to pH 3.0 ± 0.1 with |
| | triethylamine R |
| Flow | 1.5 ml/min |
| Injection Volume | 10 μl |
| Run time | 40 mins; 2 times the retention time of compound |
| | of formula II |
| Run time: | System suitability: standard solution (b) |
| Detector | 235 nm |
| Column temperature | 50° C. |
| Plate count: | minimum 10,000 |

### Impurity Profile of Compound II

| | |
|---|---|
| Column | LUNA C-8(2), (250 × 4.6 mm), |
| | 5 μm, P/N-00G-4249-EO |
| Flow | 1.5 ml/min |
| Injection Volume | 20 μl |
| Detector | 235 nm |
| Run time | 70 mins |
| Column temperature | 50° C. |

### Impurity Profile of Compound IV

| | |
|---|---|
| Column | LUNA C-8(2), (250 × 4.6 mm), |
| | 5 μm, P/N-00G-4249-EO |
| Flow | 1.0 ml/min |
| Injection Volume | 10 μl |
| Detector | 210 nm |
| Run time | 60 mins |
| Column temperature | 35° C. |

### Gradient Program

| Time | % Eluent A | % Eluent B |
|---|---|---|
| | 60 | 40 |
| 10.0 | 60 | 40 |
| 35.0 | 20 | 80 |
| 55.0 | 20 | 80 |
| 56.0 | 60 | 40 |
| 60.0 | 60 | 40 |

### Synthesis of Compound IV

### Example I(a)

In a four-necked, round-bottomed flask fitted with a thermometer pocket were added Compound II (0.207 kg, 0.20 mol) toluene (1.5 L), neopentyl glycol (0.189 kg, 1.81 mol) and pyridinium hydrobromide (0.0032 kg, 0.019 mol) at 20-25° C. to form a reaction mixture. The reaction mixture was then refluxed for 8 hours at 110-115° C. The progress of the reaction was monitored by TLC/HPLC. After completion of the reaction, the reaction mixture was cooled to an ambient temperature and washed with demineralized water (3×0.5 L). As used herein, demineralized water is water that is passed through active resin beds to remove metallic ions and filtered through a submicron filter to remove suspended impurities. The organic layer was concentrated, and the solid is suspend in ethanol (1 L), refluxed for 1 hours, cooled to 0-5° C., and filtered to afford Compound IV. Yield: 0.095 kg.

### Example I(b)

In a four-necked, round-bottomed flask fitted with a thermometer pocket were added Compound II (0.124 kg, 0.20 mol), toluene (1.5 L), neopentyl glycol (0.189 kg, 1.81 mol) and pyridinium hydrobromide (0.0032 kg, 0.019 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered to afford Compound IV. Yield: 0.094 kg.

### Example I(c)

In a four-necked, round-bottomed flask fitted with a thermometer pocket were added Compound II (0.124 kg, 0.20 mol), toluene (1.5 L), neopentyl glycol (0.189 kg, 1.81 mol) and pyridinium paratoluenesulfonate (0.005 kg, 0.02 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered to afford Compound IV. Yield: 0.092 kg.

### Example I(d)

In a four-necked, round-bottomed flask fitted with a thermometer pocket were added Compound II (0.207 kg, 0.20 mol), toluene (1.5 L), neopentyl glycol (0.189 kg, 1.81 mol) and pyridinium paratoluenesulfonate (0.005 kg, 0.02 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered to afford Compound IV. Yield: 0.088 kg.

### Example I(e)

In a four-necked, round-bottomed flask fitted with a thermometer pocket were added Compound II (0.124 kg, 0.20 mol), toluene (1.5 L), neopentyl glycol (0.189 kg, 1.81 mol) and paratoluenesulfonic acid (0.0038 kg, 0.019 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered to afford Compound IV. Yield: 0.087 kg.

### Example I(f)

In a four-necked, round-bottomed flask fitted with a thermometer pocket were added Compound II (0.207 kg, 0.20 mol), toluene (1.5 L), neopentyl glycol (0.189 kg, 1.81 mol) and paratoluenesulfonic acid (0.0038 kg, 0.019 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered t to afford Compound IV. Yield: 0.085 kg.

### Example I(g)

In a four-necked, round-bottomed flask fitted with a thermometer pocket were added Compound II (0.1 kg, 0.20 mol), toluene (1.5 L), neopentyl glycol (0.189 kg, 1.81 mol) and trimethyl silylchloride (0.0218 kg, 0.2 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered to afford Compound IV. Yield: 0.080 kg.

### Example I(h)

In a four-necked, round-bottomed flask fitted with a thermometer pocket were added Compound II (0.1 kg, 0.20 mol), toluene (1.5 L), neopentyl glycol (0.189 kg, 1.81 mol) and boron trifluoride-etherate (0.0628 kg, 0.44 mol) at 20-25° C. to form a reaction mixture. The reaction mixture was then refluxed for 8 hrs at 110-115° C. The progress of the reaction was monitored by TLC/HPLC. After completion of the reaction, the reaction mixture was cooled to an ambient temperature and washed with demineralized water (3×0.5 L). The organic layer was concentrated, and the solid is suspend in ethanol (1 L), refluxed for 1 hours, cooled to 0-5° C., and filtered to afford Compound IV. Yield: 0.082 kg.

### Example I(i)

In a four-necked, round-bottomed flask fitted with a thermometer pocket were added Compound II (0.207 kg, 0.20 mol), cyclohexane (1.0 L), neopentyl glycol (0.189 kg, 1.81 mol) and pyridinium hydrobromide (0.0032 kg, 0.019 mol) at 20-25° C. to form a reaction mixture. The reaction mixture was then refluxed for 8 hours at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.095 kg.

### Example I(j)

In a four-necked, round-bottomed flask fitted with a thermometer pocket were added Compound II (0.124 kg, 0.20 mol), cyclohexane (0.62 L), neopentyl glycol (0.189 kg, 1.81 mol) and pyridinium hydrobromide (0.0032 kg, 0.019 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.095 kg.

### Example I(k)

In a four-necked, round-bottomed flask fitted with a thermometer pocket were added Compound II (0.124 kg, 0.20 mol), cyclohexane (0.62 L), neopentyl glycol (0.189 kg, 1.81 mol) and pyridinium paratoluenesulfonate (0.005 kg, 0.02 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.093 kg.

### Example I(l)

In a four-necked, round-bottomed flask fitted with a thermometer pocket were added Compound II (0.207 kg, 0.20 mol), cyclohexane (1.0 L), neopentyl glycol (0.189 kg, 1.81 mol) and pyridinium paratoluenesulfonate (0.005 kg, 0.02 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.089 kg.

### Example I(m)

In a four-necked, round-bottomed flask fitted with a thermometer pocket were added Compound II (0.124 kg, 0.20 mol), cyclohexane (0.62 L), neopentyl glycol (0.189 kg, 1.81 mol) and paratoluenesulfonic acid (0.0038 kg, 0.019 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.088 kg.

### Example I(n)

In a four-necked, round-bottomed flask fitted with a thermometer pocket were added Compound II (0.207 kg, 0.20 mol), cyclohexane (1.0 L), neopentyl glycol (0.189 kg, 1.81 mol) and paratoluenesulfonic acid (0.0038 kg, 0.019 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.086 kg.

### Example I(o)

In a four-necked, round-bottomed flask fitted with a thermometer pocket were added Compound II (0.1 kg, 0.20 mol), cyclohexane (0.5 L), neopentyl glycol (0.189 kg, 1.81 mol) and trimethyl silylchloride (0.0218 kg, 0.2 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at at 50-70° C. The organic layer was concentrated, and the solid is suspend in ethanol (1 L), refluxed for 1 hours, cooled to 0-5° C., and filtered to afford Compound IV. Yield: 0.082 kg.

### Example I(p)

In a four-necked, round-bottomed flask fitted with a thermometer pocket were added Compound II (0.1 kg, 0.20 mol),cyclohexane (0.5 L), neopentyl glycol (0.189 kg, 1.81 mol) and boron trifluoride-etherate (0.0628 kg, 0.44 mol) at 20-25° C. to form a reaction mixture. The reaction mixture was then refluxed for 8 hrs at 80-85° C. The progress of the reaction was monitored by TLC/HPLC. After completion of the reaction, the reaction mixture was cooled and washed with demineralized water (3×0.5 L) at 50-70° C. The organic layer was concentrated, and the solid is suspend in ethanol (1 L), refluxed for 1 hours, cooled to 0-5° C., and filtered to afford Compound IV. Yield: 0.084 kg.

### Synthesis of Compound II

### Example II(a)

In a four-necked, round-bottomed flask fitted with a thermometer pocket were added Compound IV (0.045 kg, 0.0772 mol) and formic acid (0.675 L). The resulting reaction mixture was then stirred for 1 hour at 20-25° C. The progress of the reaction was monitored by TLC/HPLC. After completion of the reaction, the reaction mixture was concentrated under vacuum to afford an oil which was dissolved in methylene dichloride (MDC) (0.225 L) and washed with demineralized water (3×0.225 L). The organic layer was concentrated to an oil and crystallized from isopropyl alcohol (“IPA”) to afford Compound II. Yield 0.033 kg.

### Example II(b)

In a four-necked, round-bottomed flask fitted with a thermometer pocket were added Compound IV (0.037 kg, 0.0634 mol) and formic acid (0.555 L). The resulting reaction mixture was then stirred for 1 hour at 20-25° C. The progress of the reaction was monitored by TLC/HPLC. After completion of the reaction, the reaction mixture was concentrated under vacuum to afford an oil which was dissolved in MDC (0.185 L) and washed with demineralized water (3×0.185 L). The organic layer was concentrated to an oil and crystallized from IPA to afford Compound II. Yield 0.0265 kg.

### Example II(c)

In a four-necked, round-bottomed flask fitted with a thermometer pocket were added Compound IV (0.036 kg, 0.0617 mol) and formic acid (0.540 L). The resulting reaction mixture was then stirred for 1 hour at 20-25° C. The progress of the reaction was monitored by TLC/HPLC. After completion of the reaction, the reaction mixture was concentrated under vacuum to afford an oil which was dissolved in MDC (0.180 L) and washed with demineralized water (3×0.180 L). The organic layer was concentrated to an oil and crystallized from IPA to afford Compound II. Yield 0.026 kg.

### Example II(d)

In a four-necked, round-bottomed flask fitted with a thermometer pocket were added Compound IV (0.040 kg, 0.0686 mol) and formic acid (0.6 L). The resulting reaction mixture was then stirred for 1 hour at 20-25° C. The reaction was monitored by TLC/HPLC. After completion of the reaction, the reaction mixture was concentrated under vacuum to afford an oil which was dissolved in MDC (0.2 L) and washed with demineralized water (3×0.2 L). The organic layer was concentrated to an oil and crystallized from IPA to afford Compound II. Yield 0.0291 kg.

### Example II(e)

In a four-necked, round-bottomed flask fitted with a thermometer pocket were added Compound IV (0.070 kg, 0.120 mol) and formic acid (1.05 L). The resulting reaction mixture was then stirred for 1 hour at 20-25° C. The progress of the reaction was monitored by TLC/HPLC. After completion of the reaction, the reaction mixture was concentrated under vacuum to afford an oil which was dissolved in MDC (0.35 L) and washed with demineralized water (3×0.35 L). The organic layer was concentrated to an oil and crystallized from IPA to afford Compound II. Yield 0.051 kg.

### Example II(f)

In a four-necked, round-bottomed flask fitted with a thermometer pocket were added Compound IV (0.020 kg, 0.0343 mol) and formic acid (0.3 L). The resulting reaction mixture was then and stirred for 1 hour at 20-25° C. The progress of the reaction was monitored by TLC/HPLC. After completion of the reaction, the reaction mixture was concentrated under vacuum to afford an oil which was dissolved in MDC (0.1 L) and washed with demineralized water (3×0.1 L). The organic layer was concentrated to an oil and crystallized from IPA to afford Compound II. Yield 0.0142 kg.

### Example II(g)

In a four-necked, round-bottomed flask fitted with a thermometer pocket were added Compound IV (0.033 kg, 0.0566 mol) and acetic acid (0.495 L). The resulting reaction mixture was then stirred for 1 hour at 20-25° C. The progress of the reaction was monitored by TLC/HPLC. After completion of the reaction, the reaction mixture was concentrated under vacuum to afford an oil which was dissolved in MDC (0.165 L) and washed with demineralized water (3×0.165 L). The organic layer was concentrated to an oil and crystallized from IPA to afford Compound II. Yield 0.0234 kg

### Example II(h)

In a four-necked, round-bottomed flask fitted with a thermometer pocket was added Compound IV (0.033 kg, 0.0566 mol ) in a mixture of IPA (0.495 L) and sulfuric acid (0.0165 L). The resulting reaction mixture was then stirred for 1 hour at 20-25° C. The progress of the reaction was monitored by TLC/HPLC. After completion of the reaction, the reaction mixture was concentrated under vacuum to afford an oil which was dissolved in MDC (0.165 L) and washed with demineralized water (3×0.165 L). The organic layer was concentrated to an oil and crystallized from IPA to afford Compound II. Yield 0.024 kg.

### Example II(i)

In a four-necked, round-bottomed flask fitted with a thermometer pocket was added Compound IV (0.045 kg, 0.0772 mol) in methylene chloride (0.068 L) and formic acid (0.09 L). The resulting reaction mixture was then stirred for 4 hour at 20-25° C. The progress of the reaction was monitored by TLC/HPLC. After completion of the reaction, the reaction mixture was washed with demineralized water (3×0.225 L). The organic layer was concentrated to an oil and crystallized from ethanol to afford Compound II. Yield 0.035 kg.

What is claimed is:

1. A process for purifying (3R,4S)-4-(4-hydroxyprotected-phenyl)-1-(4-fluorophenyl)-3-[3-(4-fluorophenyl)-3-oxopropyl]azetidin-2-one having the following formula II 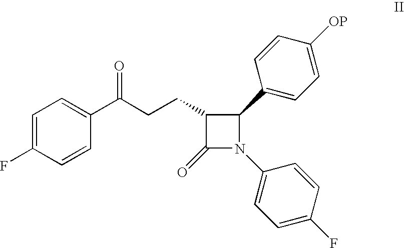
comprising the steps of:
(a) reacting the Compound of formula II with a diol derivative of formula III: 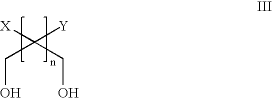
in the presence of at least one acid catalyst selected from the group consisting of: sulfonic acid, para-toluene sulfonic acid, methane sulfonic acid, benzene sulfonic acid, a C₁₋₄ tri-alkylsilyl chloride, titanium tetrachloride, titanium tetra isopropoxide, aluminum chloride, zinc chloride, and BF₃ etherate, or at least one salt of an organic base selected from the group consisting of: pyridinum hydrobromide, pyridinum hydrochloride, pyridinum hydroiodide, pyridinum paratoluenesulfonate, pyridinum methane sulfonate, pyridinum benzene sulfonate, C₁₋₄ tri-alkyl amine hydrochloride, C₁₋₄ tri-alkyl amine hydrobromide, and C₁₋₄ tri-alkyl amine hydroiodide to obtain a Compound of formula IV: 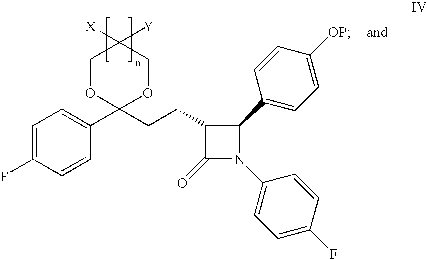
(b) adding an acid to obtain the Compound of formula II, wherein X and Y are hydrogen or a substituted or unsubstituted C₁₋₈ alkyl; n is an integer between 0 and 3; and
P is a hydroxyl protecting group.

2. The process of claim 1, wherein the diol derivative of formula III is neopentyl glycol, propane-1,3-diol, or ethylene glycol.

3. The process of claim 1, wherein the diol derivative of formula III is neopentyl glycol.

4. The process of claim 1, wherein X or Y is methyl.

5. The process of claim 1, wherein n=1.

6. The process of claim 1, wherein P is a hydroxyl protecting group selected from the group consisting of: benzyl, trimethylsilyl, para-methoxy benzyl, acetyl, and methyl.

7. The process of claim 1, wherein P is benzyl.

8. The process of claim 1, wherein the acid catalyst is selected from the group consisting of para-toluene sulfonic acid, trimethyl silyl chloride, and BF₃ etherate.

9. The process of claim 1, wherein the salt of an organic base is selected from the group consisting of pyridinum hydrobromide and pyridinum paratoluenesulfonate.

10. The process of claim 1, wherein the acid is selected from the group consisting of a mineral acid, a C₂₋₆ carboxylic acid, camphor sulfonic acid, and mixtures thereof.

11. The process of claim 1, wherein the acid is selected from the group consisting of a hydrochloric acid, sulfuric acid, phosphoric acid, hydrobromic acid, formic acid, acetic acid, propionic acid, camphor sulfonic acid, and mixtures thereof.

12. The process of claim 1, wherein the Compound of formula II is reacted with the diol derivative of formula III in the presence of an organic solvent.

13. The process of claim 1, wherein the Compound of formula II is reacted with the diol derivative of formula III in the presence of at least one organic solvent selected from the group consisting of a C₁ to C₈ halogenated hydrocarbon, a C₆ to C₁₄ aromatic hydrocarbon, and a C₁ to C₈ aliphatic hydrocarbon.

14. The process of claim 1, wherein the Compound of formula II is reacted with the diol derivative of formula III in the presence of at least one of dichloromethane, dichloroethane, or toluene.

15. The process of claim 1, wherein the acid is added to the Compound of formula IV in the presence of at least one organic solvent.

16. The process of claim 1, wherein the acid is added to the Compound of formula IV in the presence of at least one organic solvent selected from the group consisting of a C₆ to C₁₄ aromatic hydrocarbon, a C₁ to C₅ alcohol, a C₂ to C₇ ester, a C₄ to C₇ ether, and a C₁ to C₈ halogenated hydrocarbon.

17. The process of claim 1, wherein the acid is added to the Compound of formula IV in the presence of at least one organic solvent selected from the group consisting of toluene, xylene, methanol, ethanol, propanol, isopropanol, ethyl acetate, propyl acetate, tetrahydrofuran, methyl tert butyl ether, and dichloromethane.

18. The process of claim 1, wherein the acid is added to the Compound of formula IV in the presence of ethanol, propanol, or dichloromethane.

19. A process for preparing a Compound of formula II 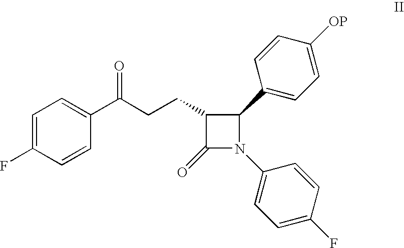
comprising adding an acid to a Compound of formula IV: 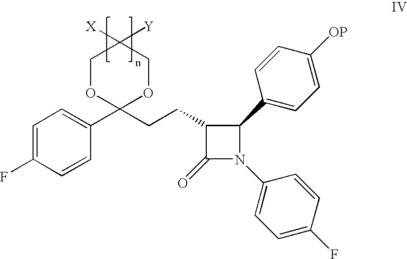
to obtain the Compound of formula II,
wherein X and Y are hydrogen or a substituted or unsubstituted C₁₋₈ alkyl; n is an integer between 0 and 3; and P is a hydroxyl protecting group.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The Compound of formula II prepared according to claim 1.

33. The Compound of formula II prepared according to claim 32 having a purity of at least about 95% by percent weight HPLC.

34. (canceled)

35. (canceled)

36. (canceled)

37. A process for preparing a Compound of formula IV: 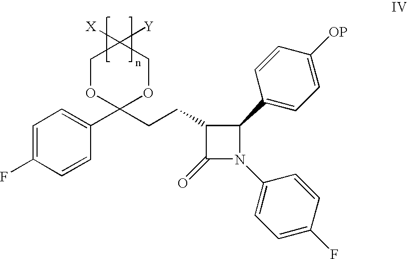
comprising reacting a Compound of formula II: 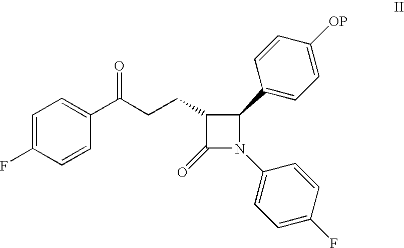
with a diol derivative of formula III: 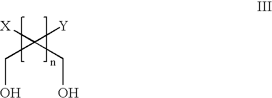
in the presence of at least one acid catalyst selected from the group consisting of: sulfonic acid, para-toluene sulfonic acid, methane sulfonic acid, benzene sulfonic acid, a C₁₋₄ tri-alkylsilyl chloride, titanium tetrachloride, titanium tetra isopropoxide, aluminum chloride, zinc chloride, and BF₃ etherate, or at least one salt of an organic base selected from the group consisting of: pyridinum hydrobromide, pyridinum hydrochloride, pyridinum hydroiodide, pyridinum paratoluenesulfonate, pyridinum methane sulfonate, pyridinum benzene sulfonate, C₁₋₄ tri-alkyl amine hydrochloride, C₁₋₄ tri-alkyl amine hydrobromide, and C₁₋₄ tri-alkyl amine hydroiodide to obtain the Compound of formula IV,
wherein X and Y are hydrogen or a substituted or unsubstituted C₁₋₈ alkyl; n is an integer between 0 and 3; and P is a hydroxyl protecting group.

38. (canceled)

39. (canceled)

40. (canceled)

41. (canceled)

42. (canceled)

43. (canceled)

44. (canceled)

45. (canceled)

46. A Compound of formula II: 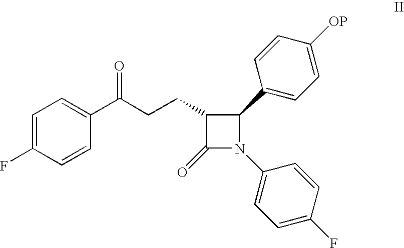
having a purity of at least about 95% by percent weight HPLC, wherein P is benzyl.

47. (canceled)

48. (canceled)

49. (canceled)

50. A Compound of formula II: 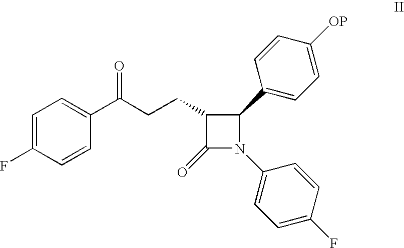
having an assay of at least about 98% by HPLC, wherein P is benzyl.

51. (canceled)

52. (canceled)

53. (canceled)

54. The compound of formula IV prepared according to claim 37.

55. A process for preparing an azetidinone compound comprising preparing the Compound of formula II according to claim 1, and converting the Compound of formula II to an azetidinone compound.

56. A process for preparing an azetidinone compound comprising converting the Compound of formula IV according to claim 37 to an azetidinone compound.

57. A process for preparing an azetidinone compound comprising converting the Compound of formula II according to claim 32 to an azetidinone compound.

58. A process for preparing an azetidinone compound comprising converting the Compound of formula IV according claim 54 to an azetidinone compound.

59. An azetidinone compound prepared according to the process of claim 55.

60. A pharmaceutical composition comprising the azetidinone compound of claim 59.

61. A method for reducing cholesterol in a mammal comprising administering a therapeutically effective amount of the azetidinone compound of claim 59.

62. A method for reducing cholesterol in a mammal comprising administering a therapeutically effective amount of the composition of claim 60.

63. The process of claim 55, wherein the azetidinone compound is ezetimibe.

64. The pharmaceutical composition of claim 60, wherein the azetidinone compound is ezetimibe.

65. The method of claim 61, wherein the azetidinone compound is ezetimibe.

66. A process for preparing an azetidinone compound comprising converting the Compound of formula II according to claim 46 an azetidinone compound.
